(19) United States
(12) Patent Application Publication
(10) Pub. No.: US 2025/0288420 A1
(43) Pub. Date: Sep. 18, 2025

(54) IMPLANT FOR FIXING A CRANIAL BONE FLAP IN A CRANIAL OPENING OF AN OSSEOUS SKULL

(71) Applicant: Aesculap AG, Tuttlingen (DE)

(72) Inventors: Jana Kuemmerlin, Regensburg (DE); Lena Vogt, Tuttlingen (DE)

(21) Appl. No.: 18/860,079

(22) PCT Filed: May 4, 2023
(86) PCT No.: PCT/EP2023/061749

§ 371 Date: Oct. 25, 2024

(30) Foreign Application Priority Data
May 4, 2022 (DE) 10 2022 204 397.9

(51) Int. Cl. A61F 2/28; A61F 2/30

(52) U.S. Cl. CPC A61F 2/2875; A61F 2/2846; A61F 2/30771; A61F 2002/285; A61F 2002/30069; A61F 2002/30677; A61F 2002/3092

(57) ABSTRACT

An implant for fixing a cranial bone flap in a cranial opening of an osseous skull includes a flexible mesh structure having webs and openings formed between the webs. The flexible mesh structure is made of a plastic material and is designed to be fastened to the bone flap and to the osseous skull.

### CROSS-REFERENCE TO RELATED APPLICATIONS

This application is the United States national stage entry of International Application No. PCT/EP2023/061749, filed on May 4, 2023, and claims priority to German Application No. 10 2022 204 397.9, filed on May 4, 2022. The contents of International Application No. PCT/EP2023/061749 and German Application No. 10 2022 204 397.9 are incorporated by reference herein in their entireties.

### FIELD

The invention relates to an implant for fixing a cranial bone flap in a cranial opening of a bony skull.

### BACKGROUND

Neurosurgical procedures on the brain are generally performed through an opening in the bony skull. The production of such a cranial opening is also referred to as a craniotomy. In a craniotomy, part of the skull bone is separated from the bony skull by trepanation and usually with the help of a craniotome. The detached part is also referred to as a bone flap or bone fragment. After the procedure is finished, the bone flap is repositioned and fixed in the cranial opening produced beforehand.

To fix the bone flap, implants in the form of what are referred to as clamp systems are known. For example, a clamp system under the designation CranioFix®2 from Aesculap AG, Tuttlingen, Germany, is known. The known clamp system has an upper disk element, a lower disk element, and a rod element which movably connects the two disk elements to each other. To fix the bone flap, firstly the lower disk element is placed on the internal lamina of the skull cap, the rod element being aligned approximately parallel to the axial direction of the cranial opening. The bone flap is repositioned in the cranial opening, its inner side being placed onto the lower disk element. Here, the rod element extends from the skull inner side, through the craniotomy gap formed between the bone flap and the surrounding skull bone, to the skull outer side. For final fixing, the upper disk element is moved closer to the lower disk element along the rod element with the aid of several snap elements and is pressed with a defined force into the end position. In the final fixing state, the two disk elements are pressed both fixedly onto the outer side and fixedly onto the inner side of the bone flap and of the surrounding skull bone in the axial direction of the cranial opening.

Various aspects of the known clamp system can be considered disadvantageous: The arrangement of the lower disk element in the epidural space can under certain conditions result in irritation of the dura mater or even lead to an epidural hematoma. The axial clamping of the disk elements carries the risk of unintended pressure being applied to the dura mater. In order to avoid unintended application of compressive force, a special tool for holding the rod element is provided in the case of CranioFix®2. The additionally required tool can make handling more difficult. In addition, the disk elements remain permanently on the patient's skull after fixation. This is particularly disadvantageous in the case of the upper disk element in cosmetically critical regions, for example in a frontal craniotomy. Since the upper and the lower disk element must lie on the skull bone and the bone flap as flat as possible for satisfactory fixing, fixing is often not possible, or not satisfactorily possible, in the case of considerable curvatures or thickness gradients. Considerable curvatures can moreover result in the upper disk element protruding from the skull outer side, which can result in inadequate cosmetic results and/or scalp irritation.

### SUMMARY

It is an object of the invention to provide an implant of the type mentioned in the introduction which offers advantages over the prior art. In particular, the intention is to at least partially overcome or mitigate the disadvantages involved in the prior art.

The implant according to the invention has a flexible mesh structure with webs and openings formed between the webs, wherein the flexible mesh structure is made of a plastic material and is arranged for fastening to the bone flap and to the bony skull. The solution according to the invention enables improved medical and cosmetic results and also particularly simple application and revision. The flexible mesh structure extends in a planar fashion and therefore has a small thickness in comparison to conventional clamping systems or the like. The relatively small thickness enables improved cosmetic results, since the flexible mesh structure is less visible under the patient's scalp. Thus, the implant according to the invention is preferably applicable to cosmetically critical regions of the skull, for example in a frontal craniotomy. Since the mesh structure is flexible, its shape can be adapted particularly easily to a given skull geometry of the patient. Protrusion of the implant is avoided even in more convexly or concavely curved regions of the skull. The thin yet flexible design also prevents irritation of the scalp. This is beneficial from a medical point of view. The flexible and thin design, in conjunction with the production from said plastic material, also enables particularly simple application by the operating surgeon. For example, the flexible mesh structure can be cut to a size and geometry suitable for the respective craniotomy, without great effort and with simple tools, for example scissors or a scalpel, as a result of which simplified and particularly versatile use is achieved. This is in contrast to known metal implants, which usually need to be provided in different sizes and/or geometries and do not permit easy adjustment of the size and/or geometry by the end user. The flexible mesh structure is designed for fastening to the bone flap and to the bony skull, wherein the fastening can be carried out in particular in an integrally bonded manner by means of a suitable adhesive or in a form-fitting and/or force-fitting manner by means of suitable fastening elements. For its fastening, the flexible mesh structure is applied to the outer side of the skull and/or an outer side of the bone flap and fastened. Removal of the implant for revision purposes is therefore straightforward. In particular, no special instrument is required for this purpose. If the flexible mesh structure is to be bonded on, it can be removed from the bone flap and the bony skull for revision in the manner of a plaster. In one embodiment, the webs and/or openings of the mesh structure are arranged regularly, in particular at regular intervals and/or in a regular pattern. In a further embodiment, the webs and/or openings are arranged irregularly. In one embodiment, the openings have an opening width of 2 μm to 5 mm. In a further embodiment, the openings each have an opening width in the range of 2 μm to 1 mm. In a further embodiment, the openings each have an opening width in the range of 2 μm to 20 μm. The plastic material is at least partially absorbable in one embodiment, preferably fully absorbable. In a further embodiment, the plastic material is not absorbable. The plastic material is biocompatible. Biocompatible plastics for medical applications are known to a person skilled in the art. In one embodiment, the implant consists of the flexible mesh structure. In further embodiments, the implant has a multi-layer configuration, wherein the flexible mesh structure forms a layer of the multi-layer configuration.

In one embodiment, the flexible sheet-like structure is translucent. In other words, the flexible mesh structure is transparent. In this way, artefacts are avoided when using imaging techniques. If the flexible mesh structure is provided to be integrally bonded to the bone flap and to the bony skull surrounding the cranial opening, the translucent properties allow the use of an adhesive with radiation-induced curing.

In a further embodiment, the flexible mesh structure has a thickness of 0.2 mm to 0.5 mm. On the one hand, this allows particularly good cosmetic results to be achieved, since the flexible mesh structure is not visible or barely visible under the patient's scalp on account of the small thickness. On the other hand, a thickness within said value range ensures sufficient mechanical properties.

In a further embodiment, the flexible mesh structure has isotropic elasticity. Isotropic elasticity means that the flexible mesh structure has directionally independent elastic properties in its plane of extension. This allows the flexible mesh structure to be adapted to the given skull geometry of the patient in a further improved manner during application.

In a further embodiment, the webs are arranged crossing over without knots, and the flexible mesh structure is in the form of a knitted fabric or a woven fabric. Without knots means that the webs of the flexible mesh structure do not have points of intersection firmly connected to one another. The flexible mesh structure is thus particularly easy to adapt to the given shape of the bone flap and of the surrounding bony skull. This further simplifies the application of the implant. In this embodiment, the flexible mesh structure is a knitted fabric or a woven fabric. Alternatively, production by means of electrospinning or additive manufacturing processes is conceivable. In an alternative embodiment, the webs are firmly connected to one another at their respective points of intersection. In particular, simplified cutting to size can be achieved in this way. Falling apart of the flexible mesh structure is avoided.

In a further embodiment, the plastic material contains polypropylene and/or polyglycolic acid. The inventors have found that this can bring particular advantages. On account of their good biocompatibility, polypropylene and polyglycolic acid are already used for the production of medical suture material. This shows their good suitability from a mechanical point of view. While polypropylene is not absorbable, polyglycolic acid can be absorbed hydrolytically by the body within about 60 to 90 days. In addition, polypropylene has transparent properties, which can simplify implantation. The group of polylactic acids, in particular with transparent properties, can alternatively or additionally be a component of the plastic material. In further embodiments, plastics of natural origin are conceivable, such as cellulose, silk, fibroin, gelatin or collagen, in a mixture with polypropylene and/or polyglycolic acid, in particular for promoting the biocompatible properties.

In a further embodiment, the plastic material contains a non-metallic additive, preferably glass fibers and/or ceramic particles. These additives can increase mechanical strength and/or osseointegration.

In a further embodiment, the implant has at least one medical active substance, which is applied in the form of an active substance coating to the flexible mesh structure and/or is introduced in the form of an active substance filling into the openings. Alternatively or additionally, it can be said that the medical active substance is applied to the mesh structure and/or introduced into the mesh structure. The medical active substance is, for example, an antibiotic, an analgesic, growth factors or the like. By virtue of the sheet-like extent of the flexible mesh structure, locally excessive active substance concentrations are avoided, and particularly advantageous release of the active substance is possible. In particular, improved regeneration of the periosteum is possible. In one embodiment, the medical active substance in the form of said active substance coating is applied to the flexible sheet-like structure, in particular to its underside and/or top side. In a further embodiment, the medical active substance is alternatively or additionally introduced in the form of an active substance filling into the openings of the flexible mesh structure.

In further embodiment, the implant has an adhesive, which is applied in the form of an adhesive layer to the flexible mesh structure and/or is introduced in the form of an adhesive filling into the openings. In this embodiment of the invention, it is possible to dispense with the use of a separate adhesive product and/or separate fastening elements. The adhesive, in the form of the adhesive layer and/or of the adhesive filling, is a constituent part of the implant. In one embodiment, the adhesive is at least partially absorbable, preferably fully absorbable. In a further embodiment, the adhesive is not absorbable. All biocompatible adhesives suitable for medical applications are suitable for use. Such adhesives are known to a person skilled in the art. If the implant is to have a medical active substance according to the preceding embodiment, this substance can be integrated into the adhesive layer and/or adhesive filling. On the one hand, the adhesive serves to fasten the flexible mesh structure to the bone flap and to the surrounding bony skull. On the other hand, the adhesive serves to mechanically stabilize the flexible mesh structure. Preferably, the adhesive is designed to cure by means of UV radiation. Alternatively or additionally, further curing mechanisms are conceivable, for example through moisture, heat or other physical and/or chemical mechanisms of action.

In a further embodiment, the adhesive is a medical tissue adhesive which cures under the influence of UV radiation. The inventors have found that such an adhesive offers particular advantages in connection with the present use.

In a further embodiment, the implant has at least one peel-off first protective layer, which is applied at least indirectly to an underside of the flexible mesh structure, and/or a peel-off second protective layer, which is applied at least indirectly to a top side of the flexible mesh structure. The first protective layer and/or the second protective layer is peeled off prior to implantation of the implant. The first protective layer serves to protect the underside of the flexible mesh structure. The second protective layer serves to protect the top side of the flexible mesh structure. If the flexible mesh structure has no active substance layer, adhesive layer and/or other layer on its underside and/or top side, the first protective layer and/or second protective layer is applied directly to the flexible mesh structure. Otherwise, the corresponding protective layer covers the flexible mesh structure only indirectly and is applied directly to one of said layers. The first protective layer and/or second protective layer is preferably an in particular smooth protective film. The protective film is preferably made of a suitable plastic material, for example PTFE.

The invention also relates to a kit having an implant and an adhesive, which is arranged for bonding the flexible mesh structure to the bone flap and to the bony skull. In this embodiment, the adhesive is made available as a separate product and, together with said implant, forms a kit for fixing a cranial bone flap. For the purpose of fastening the flexible mesh structure, the adhesive is applied to the underside and/or top side of the latter before its actual application. Adhesives suitable for this purpose are known to a person skilled in the art. In one embodiment, the adhesive is a spray adhesive, which can be sprayed onto the flexible mesh structure.

The invention also relates to a kit having an implant and a plurality of fastening elements, which are arranged for the force-fitting and/or form-fitting fastening of the flexible mesh structure to the bone flap and to the bony skull. In one embodiment, the plurality of fastening elements are pins. Alternatively, the fastening elements can be screws, hooks or the like. In one embodiment, the pins are degradable with pre-drilling. In a further embodiment, the pins are non-degradable and self-cutting. The fastening elements are inserted into the outer side of the bone flap, and into the bony skull surrounding the cranial opening, before the actual application of the implant. The fastening elements used form anchor points for the flexible mesh structure, which can be hooked onto the fastening elements by means of its openings and braced.

### BRIEF DESCRIPTION OF THE DRAWING FIGURES

Further advantages and features of the invention will emerge from the following description of preferred exemplary embodiments of the invention that are illustrated by means of the drawings.

FIG. 1 shows a schematic and highly simplified perspective view of a patient's head, the patient's bony skull having a cranial opening closed with a bone flap,

FIG. 2 shows the region of the cranial opening in a schematic plan view, wherein the bone flap is fixed using an embodiment of an implant according to the invention,

FIG. 3 shows the region of the cranial opening in a schematically simplified sectional view,

FIG. 4 shows a perspective exploded view of the implant according to FIGS. 2 and 3, in a state in which it is provided with two protective films,

FIG. 5 shows a schematic and highly simplified section through a layer structure of the implant according to FIG. 4,

FIG. 6 shows a schematic view of an embodiment of a kit according to the invention,

FIG. 7 shows a schematic view of a further embodiment of a kit according to the invention, and

FIG. 8 shows an example of an application situation using the kit according to FIG. 7 in a view according to FIG. 3.

### DETAILED DESCRIPTION

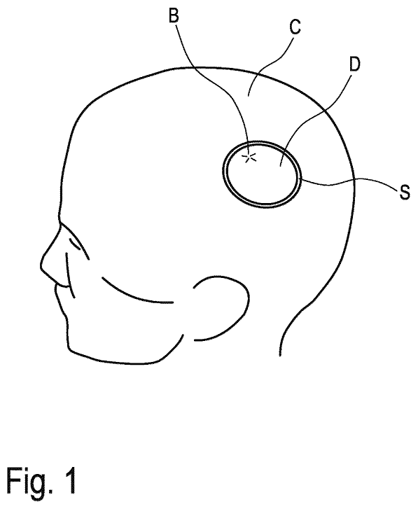

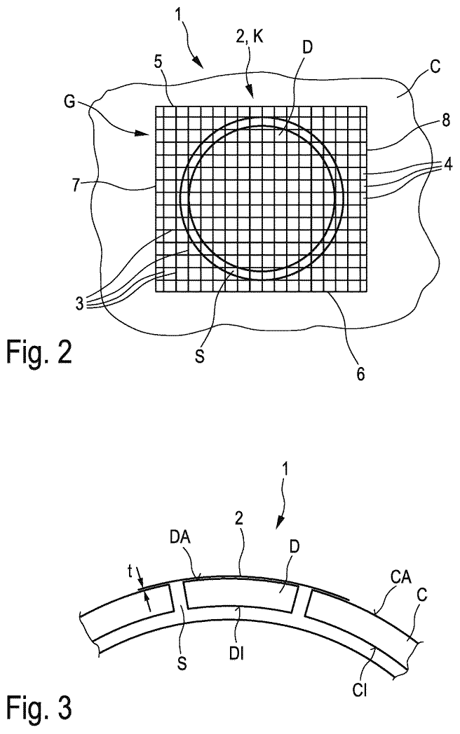

According to FIG. 1, a patient's head has a cranial opening B. The cranial opening B extends from a skull outer side CA, through the bony skull C of the patient, to a skull inner side CI (see FIG. 3). The cranial opening B extends axially through the bony skull C and is radially bordered by same.

The cranial opening B can in principle be caused by an accident or some other trauma. In the present case, the cranial opening B is the result of a craniotomy and serves as a surgically produced access for a neurosurgical procedure on the patient's brain.

During such a craniotomy, the bony skull C is opened by mechanically detaching a bone flap D. This can be performed, for example, by means of trepanation and a craniotomy. Here, the bone flap D is detached from the bony skull C to form an annular gap S. The annular gap S is also referred to as a craniotomy gap. The bone flap D can also be referred to as bone part, bone fragment or bone segment.

After the bone flap D has been detached, it can be temporarily removed from the rest of the bony skull so as to form the actual cranial opening B. After the procedure on the brain has been completed, the bone flap D is repositioned and fixed in the opening B.

According to FIG. 2, an implant 1 for fixing the cranial bone flap D in the cranial opening B is provided. The implant 1 has a flexible sheet-like structure 2 with webs 3 and with openings 4 formed between the webs. Furthermore, the flexible sheet-like structure 2 is made of a plastic material K and is configured, in a manner to be described in more detail, for fastening to the bone flap D and to the bony skull C bordering the opening B.

In the situation shown with reference to FIGS. 2 and 3, the flexible mesh structure 2 spans the bone flap D, the annular gap S and the region of the bony skull C surrounding the opening B. The flexible mesh structure 2 rests on an outer side DA of the bone flap D and the outer side CA of the bony skull C. The fastening of the flexible mesh structure 2 to the bone flap D on the one hand and to the bony skull C on the other hand is different in different embodiments and can be carried out, for example, by cohesive bonding by means of a suitable adhesive and/or using suitable fastening elements.

The arrangement of the webs 3 and/or openings 4 shown in particular in FIG. 2 is an example. In embodiments not shown in the figures, the webs and/or openings are arranged differently for this purpose; an irregular arrangement may be provided in particular. The same applies to the shown opening widths of the openings 4. These too are given purely as an example.

In the present case, the flexible mesh structure 2 has a rectangular, in particular square, shape with opposite pairs of outer edges 5, 6, 7, 8. The design shown is only an example. In embodiments not shown in the figures, the flexible mesh structure is round, oval or otherwise adapted to a geometry of the bone flap that is to be fixed.

As is shown in particular in FIG. 3, the flexible mesh structure 2 is thin compared to the involved bone structures C, D. In the present case, the flexible mesh structure 2 has a thickness t of 0.4 mm. In embodiments not shown in the figures, the thickness of the flexible mesh structure ranges between 0.2 mm and 0.5 mm. By virtue of the low thickness and the production from the plastic material K, the flexible mesh structure 2 can be easily cut to size and its dimensions adapted to the bone flap D that is to be fixed. In addition, on account of its properties, the mesh structure 2 can be easily adapted in shape to a curvature or other geometry of the involved bone structures C, D.

In the embodiment shown, the flexible mesh structure 2 is a woven fabric G, wherein the webs 3 are arranged crossing over without knots. In this respect, it is also possible to refer to a knot-free mesh. In an embodiment not shown in the figures, the webs are firmly connected to one another at their respective points of intersection, in which case it is also possible to refer to a knotted mesh.

In an embodiment not shown in the figures, the flexible mesh structure 2 is not in the form of a woven fabric, but instead a knitted fabric, a nonwoven or the like.

As is shown in particular in FIG. 2, the flexible mesh structure 2 has translucent properties. As a result, the bone flap D, the annular gap S and the regions of the bony skull C arranged below the flexible sheet-like structure 2 can be viewed through the flexible sheet-like structure 2. In particular, this permits precise positioning and is associated with further advantages to be described in more detail.

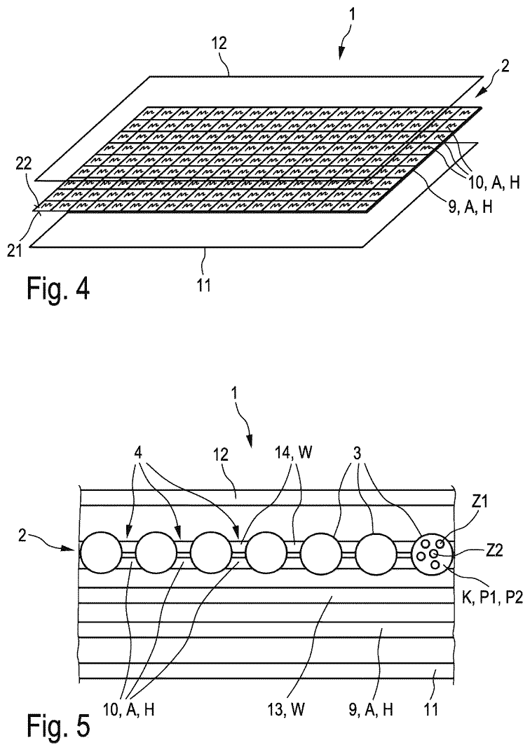

In the embodiment shown in FIGS. 2 to 5, the flexible mesh structure 2 is fastened by cohesive bonding to the bone structures C, D. For this purpose, the implant 1 has an adhesive A. The adhesive A is applied in the form of an adhesive layer 9 to the flexible sheet-like structure 2 and additionally introduced in the form of an adhesive filling 10 into the openings 4 (see FIG. 5). In embodiments not shown in the figures, the adhesive A is provided either in the form of the adhesive layer or of the adhesive filling. In the present case, the adhesive A is a medical tissue adhesive H which cures under the influence of UV radiation. As a result of the translucent properties of the flexible mesh structure 2, the tissue adhesive H can be cured particularly easily, since the UV radiation required for this purpose can be radiated through the flexible mesh structure 2.

In the embodiment shown, the implant 1 also has a medical active substance W. The medical active substance W is present in the form of an active substance layer 13 on the flexible mesh structure 2 and additionally introduced in the form of an active substance filling 14 into the openings 4. The equipping of the implant 1 with the medical active substance W is optional and does not therefore apply in all embodiments. In addition, it is possible that the active substance layer 13 and the adhesive layer 9 are as it were combined to form one layer. In other words, the active substance W can be part of the adhesive layer 9, or vice versa. Against this background, the arrangement of the layers 13, 9 shown in FIG. 5 is also to be regarded as schematic and highly simplified.

Furthermore, the implant 1 in the present case has a first protective layer 11 and a second protective layer 12. The first protective layer 11 is applied at least indirectly to an underside 21 of the flexible mesh structure 2. The second protective layer 12 is applied at least indirectly to a top side 22 of the flexible mesh structure 2. Indirectly signifies that the respective protective layer 11, 12 can be applied to further layers, depending on the layer structure, and thus indirectly to the respective side of the flexible mesh structure 2. In the present case, the second protective layer 12 is applied directly to the top side 22. The first protective layer 11 in the present case is applied directly to the adhesive layer 9 and thus indirectly to the underside 21.

Both protective layers 11, 12 are peelable and are removed before the application of the implant 1. In the present case, the protective layers 11, 12 are films made of a plastic material suitable for this purpose. The plastic material is PTFE in the embodiment shown.

FIG. 5 shows schematically that the plastic material K in the present case is provided with a non-metallic additive Z1, Z2. The additive Z1, Z2 in the present case is a mixture of glass fibers Z1 and ceramic particles Z2. The equipping of the plastic material K with said additive is optional and is intended to improve the mechanical properties and/or osseointegration of the flexible mesh structure 2.

The plastic material K in the present case is a composite of polypropylene P1 and polyglycolic acid P2.

To fix the bone flap D by means of the implant 1, the first protective layer 11 is first peeled off. Thereafter, the flexible mesh structure 2 is pressed, with the adhesive layer 9 to the front, onto the outer side DA of the bone flap D. Alternatively, the implant 1 can be placed, with the second protective layer to the front, onto a flat base. Thereafter, the bone flap D is pressed with the outer side DA onto the underside 21 of the flexible mesh structure 2, more precisely onto the adhesive layer 9 arranged there. Thereafter, the arrangement of implant 1—without first protective layer 11—and bone flap D is repositioned in the region of the cranial opening B. After repositioning is complete, the regions of the flexible mesh structure 2 that protrude laterally over the bone flap D and over the annular gap S are pressed onto the outer side CA of the surrounding bony skull C.

Thereafter, the curing of the tissue adhesive G takes place, it being possible for the second protective layer 12 to be removed either before or after the curing.

In the embodiment shown in FIGS. 2 to 5, the adhesive A is part of the implant 1. Alternatively, the adhesive A can be provided as a separate product.

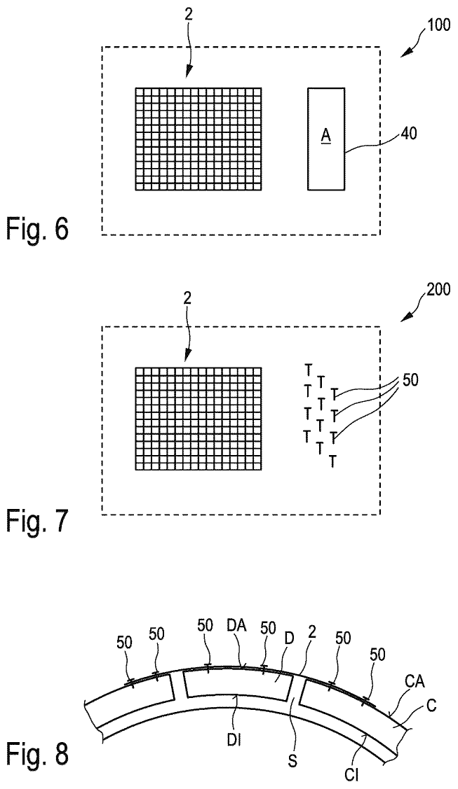

FIG. 6 shows a kit 100 having a flexible mesh structure 2 and having the adhesive A, the latter being provided as a separate product 40. The separate product 40 can be, for example, a container filled with the adhesive A, in the form of a tube or the like. The adhesive A is applied to the flexible mesh structure 2 before application of the latter.

FIG. 7 shows a further kit 200 having a flexible mesh structure 2 and having a plurality of fastening elements 50. The fastening elements 50 are configured for fastening the flexible sheet-like structure 2 to the bone structures C, D (see FIG. 8). The fastening elements 50 in the present case are pins, which are each inserted into the outer side DA of the bone flap D or into the outer side CA of the surrounding bony skull C. In the inserted state, the fastening elements 50 form anchoring points as it were, to which the flexible mesh structure 2 can be anchored.

As regards further features of the flexible mesh structure 2 contained in the kits 100, 200, reference is made expressly to the foregoing.

1-13. (canceled)

14. An implant for fixing a cranial bone flap in a cranial opening of a bony skull, the implant comprising a flexible mesh structure with webs and openings formed between the webs, the flexible mesh structure being made of a plastic material and configured to be fastened to the cranial bone flap and to the bony skull.

15. The implant according to claim 14, wherein the flexible mesh structure is translucent.

16. The implant according to claim 14, wherein the flexible mesh structure has a thickness of 0.2 mm to 0.5 mm.

17. The implant according to claim 14, wherein the flexible mesh structure has isotropic elasticity.

18. The implant according to claim 14, wherein the webs are arranged crossing over without knots.

19. The implant according to claim 14, wherein the flexible mesh structure comprises a knitted fabric or a woven fabric.

20. The implant according to claim 14, wherein the plastic material contains polypropylene and/or polyglycolic acid.

21. The implant according to claim 14, wherein the plastic material contains a non-metallic additive.

22. The implant according to claim 14, wherein the plastic material contains glass fibers and/or ceramic particles.

23. The implant according to claim 14, further comprising at least one medical active substance applied as an active substance coating to the flexible mesh structure.

24. The implant according to claim 14, further comprising at least one medical active substance introduced as an active substance filling into the openings.

25. The implant according to claim 14, further comprising an adhesive applied as an adhesive layer to the flexible mesh structure.

26. The implant according to claim 14, further comprising an adhesive filling into the openings.

27. The implant according to claim 14, further comprising a medical tissue adhesive that cures under UV radiation.

28. The implant according to claim 14, further comprising at least one of:
a first peel-off protective layer applied at least indirectly to an underside of the flexible mesh structure; and
a second peel-off protective layer applied at least indirectly to a top side of the flexible mesh structure.

29. A kit comprising:
the implant according to claim 14; and
an adhesive arranged for bonding the flexible mesh structure to the cranial bone flap and to the bony skull.

30. A kit comprising:
the implant according to claim 14; and
a plurality of fastening elements configured for force-fitting and/or form-fitting fastening of the flexible mesh structure to the cranial bone flap and to the bony skull.
